(19) United States
(12) Patent Application Publication
(10) Pub. No.: US 2005/0267502 A1
(43) Pub. Date: Dec. 1, 2005

(54) Disposable safety cutting tool

(76) Inventor: Mark N. Hochman, Lake Success, NY (US)

Correspondence Address: Tiberiu Weisz, Esq.;Gottlieb, Rackman & Reisman, P.C., 270 Madison Avenue, New York, NY, 10016-0601, US

(21) Appl. No.: 10/742,053

(22) Filed: Dec. 18, 2003

(51) Int. Cl. A61B 17/32

(52) U.S. Cl. 606/167

(57) ABSTRACT

A safety scalpel is provided that includes a tubular housing, a coupling reciprocating within the housing and holding a cutting head or blade and a control member used to selectively extend said cutting head outwardly of the housing and retracted into the housing. A biasing spring is used to hold the cutting head in a retracted position. In one embodiment a tubular groove on an inner surface of the housing may be used to simultaneously translate the cutting head and rotate it about a longitudinal axis of the housing. The cutting head can be immobilized within the housing by jamming the control tab or by removing the control tab from the housing.

### BACKGROUND OF INVENTION

1. Field of Invention

This invention pertains to a safety bladed device, safety scalpel or other similar cutting tool including a handle and a cutting head, such as a blade, that is selectively extended from the handle and then retracted after use. The total package (i.e. the handle and the cutting head) can be disposed. Alternatively, the cutting head can be disposed and the handle can be used after sterilization. In one embodiment, the cutting head rotates about a longitudinal axis as it is extended.

2. Description of the Prior Art

Scalpels are used in various surgical procedures to make an incision through the skin of a patient, or to performing other cutting/slicing operations in a patient's body. Most bladed devices of this nature are “flat” in design in which holding the bladed instrument proves to be difficult to perform a curving or rounding incisions thus resulting in a less then ideal incision design in many instances. Incisions can therefore be jagged and irregular. A “flat” design handle requires greater wrist movement to compensate for the handle design which can lead to awkward positioning of the hand during critical surgical movements.

During most of these procedures, the scalpel is in contact with body tissues as well as body fluids. Moreover, inherently scalpels have sharp edges that may cause pricks, cuts, scratches and other similar problems to the health care provider handling the scalpel, or others. Any cutaneous cut is undesirable because it could be painful, can result in bleeding and scarring. Even a small cut may take a long time to heal and be extremely inconvenient. Moreover, once used on a patient, the blade could be contaminated. Of course, in today's environment there is a great deal of concern regarding any cuts or punctures to the skin of a health care provider that can cause contamination.

Some studies indicate that in an operating room 7% of injuries are associated with surgery instruments (i.e., scalpels) and that 39% of such injuries are self-inflicted while a full 61% are inflicted by users of scalpels on co-workers. It is believed that most such injuries occur while the scalpel is being mounted or removed, while the scalpel is being passed from one user to another, or during the actual surgical procedure itself.

Therefore, it has been recommended that safety scalpels be used which are provided with a safety means that is positioned over its cutting edge when not in use. Generally present safety scalpels are available in one of two configurations. One configuration comprises a handle holding a fixed blade that extends longitudinally outwardly of the handle. A shield is also attached to the handle. The shield has a slot and is selectively slidable between two positions. In one position the shield is retracted over the handle with the fixed blade extending through the slot. In the second position, the shield is disposed over the blade so that the blade cannot be touched and it will be a health threat. A finger disposed on the shield is manipulated by the user to secure or lock the shield in either position. Safety scalpels of this type are available from BD Medical Systems of Franklin Lakes N.J. 07417 under the name of BD Bard-Parker™. The second configuration consists of a thin handle, with the blade being selectively slidable in and out of the handle, resembling essentially a conventional box cutter. Safety scalpels are available, e.g., from Deroyal of Knoxville, Ten., under the name of Deroyal Retractable Safety Scalpels; and Persona Medical, a division of the American Safety Razor Company, Verona, Va.

These safety scalpels are difficult to use and they may not provide sufficient safety.

Commonly assigned U.S. Pat. No. 6,428,517 discloses a safety syringe.

Biopsy punches are used to extract a tissue sample from a body organ and have many of the same disadvantages as scalpels.

### OBJECTIVES AND SUMMARY OF THE INVENTION

An objective of the present invention is to provide a safety scalpel or other cutting tool that can be used by a surgeon or other professionals easily and effectively.

Another objective is to provide a safety scalpel that can be locked effectively and substantially permanently in a configuration in which its cutting head is fully enclosed, thereby eliminating the chance of any contamination.

Yet another objective is to provide a safety scalpel including a cutting head that is rotatable as it is advanced and/or retrieved from a handle.

Yet a further objective is to provide a disposable scalpel using an interchangeable modular system.

Other objectives and advantages of the invention will become apparent from the following description.

Briefly, a cutting tool constructed in accordance with this invention, includes a handle forming a housing that in one aspect of the invention is rounded or approximately cylindrical. A coupling disposed inside the housing and a cutting head attached to the coupling. The coupling is arranged and constructed to selectively advance the cutting head out of the handle, preferably under the control of the operator, and once an incision, a puncture or a cut is completed, to retract it back into the handle.

In one embodiment, of the invention, a spring is provided in the housing which is arranged to bias the cutting head toward a preselected position within the housing. Preferably the cutting head is biased toward its retracted position so that if the cutting head is advanced to extend outwardly of the housing and then released, it automatically returns to the retrieved position within the housing thereby reducing the likelihood that the cutting head pricks, cuts or otherwise injures an operator, the patient, or any one else. Locking members are also provided which are used to secure the cutting head in at least one of its positions.

The coupling may be arranged and constructed to cooperate with the housing so that as the cutting head is advanced out of the housing, it is automatically and simultaneously rotated about its longitudinal axis. Typically, the cutting head may be rotated by about 20-270 degrees. Preferably the cutting head is rotated by about 180 degrees. This may be accomplished, for instance, by providing a helical groove on an inner surface of the housing and a boss on the coupling. As the coupling moves through the housing, its boss rides through the groove and this caming action causes the coupling, and the cutting head to rotate.

A control means, such as a button movable around the housing, is used to move the cutting head between its positions. Advantageously when at least a portion of this button is removed, the cutting head is no longer movable and is preferably trapped in the retracted position. In another version such button can be manipulated to obstruct the movement of the cutting head between its positions by becoming a direct interference to the movement of the cutting head, (thus jamming the movement).

The cutting head may be a relatively flat blade provided with a cutting edge, or may comprise a thin sleeve that may be used to obtain biopsies.

### BRIEF DESCRIPTION OF THE FIGURES

FIG. 1 shows a sectional somewhat diagrammatic view of a safety cutting tool constructed in accordance with this invention with a blade in an extended position;

FIG. 2 shows a sectional view similar to FIG. 1 with the blade retracted;

FIG. 3 shows a sectional view of the blade and a spring used to bias the blade toward a retracted position;

FIG. 4 shows a sectional view of the blade and the spring in the extended position;

FIG. 5 shows an embodiment of the invention which allows the blade to rotate about its axis, the blade being in a retracted position;

FIG. 6 shows a view similar to FIG. 5 with the blade being in the extended position;

FIG. 7 shows an orthogonal view of a tubular sleeve for obtaining biopsies and used for the scalpel of FIGS. 1-6; and

FIG. 8 shows an orthogonal exploded view of another embodiment.

### DETAILED DESCRIPTION OF THE INVENTION

Referring now to the drawings, a cutting tool such as a safety scalpel 10 constructed in accordance with this invention includes a handle 20 and a cutting head such as a blade 22 attached to the handle 20.

As shown in FIG. 1, the handle 20 includes in accordance with this invention a tubular housing 24 having a circular or other outer cross section, an internal wall 26, an external wall 28, and two ends 30, 32. Handle 20 further includes an elongated stem 34 provided to balance the handle and make it easier to hold. The stem 34 may be glued to the housing 24, or may be sized and shaped to form an interference fit therewith whereby it can be inserted into the housing 24 or removed at will. The stem 34 may be omitted.

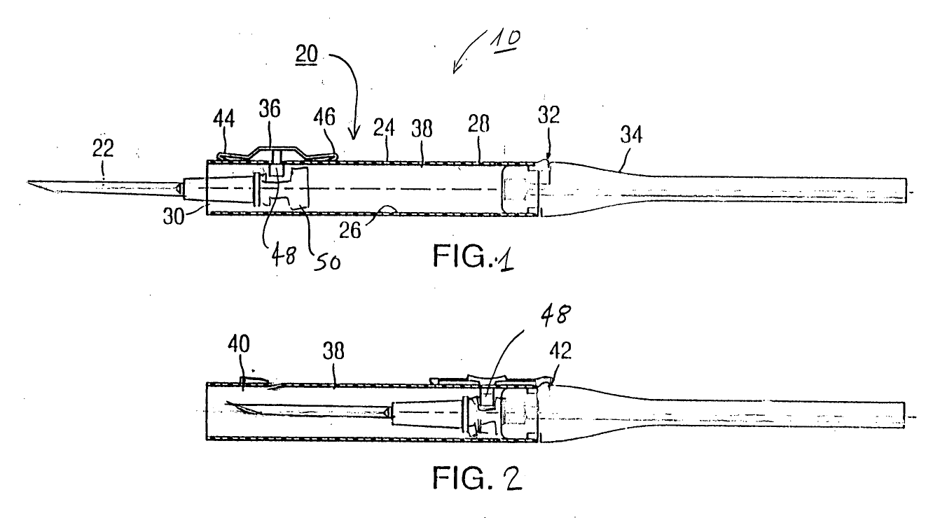

Mounted on housing 24 there is a button 36 slidable in a longitudinal slot 38 extending through the housing. Housing 24 is provided with a stop 40 at the forward end of slot 38. The rear end 42 of the housing 24 serves as a rear stop for button 36. Alternatively, a stop similar to stop 40 may be provided on the external wall 28 of housing 24. Button 36 includes two spring arms 44, 46. These arms extend in the longitudinal axis of the housing 24 and are turned upward, or away from surface 28 at their tips. These arms 44, 46 are somewhat flexible and cooperate with the stop 40 and end wall 42 to limit the movement of button 36 in slot 38. More particularly, as button 36 is reciprocated by an operator in slot 38, when the button reaches its forward most position, the arm 44 rides over the stop 40 and then snaps radially inward to form an interference fit with the stop 40. Once in this position (shown in FIG. 1) corresponding to the extended position of the blade 22, the button 36 is locked into this position. The button 36 can be released by pressing it downward toward the housing 24, thereby causing arms 44, 46 to flex upward. The button 36 can then move backward, away from the front opening 30 because the arm 44 clears the stop 40. When the button 36 is moved backwards toward stem 34, when its rear arm 46 engages the rear wall 42, the button 36 is locked into the rear position, in which the blade 22 is in its fully retracted position, as indicated in FIG. 2.

Button 36 further includes a pin 48 extending downwardly or radially through slot 38.

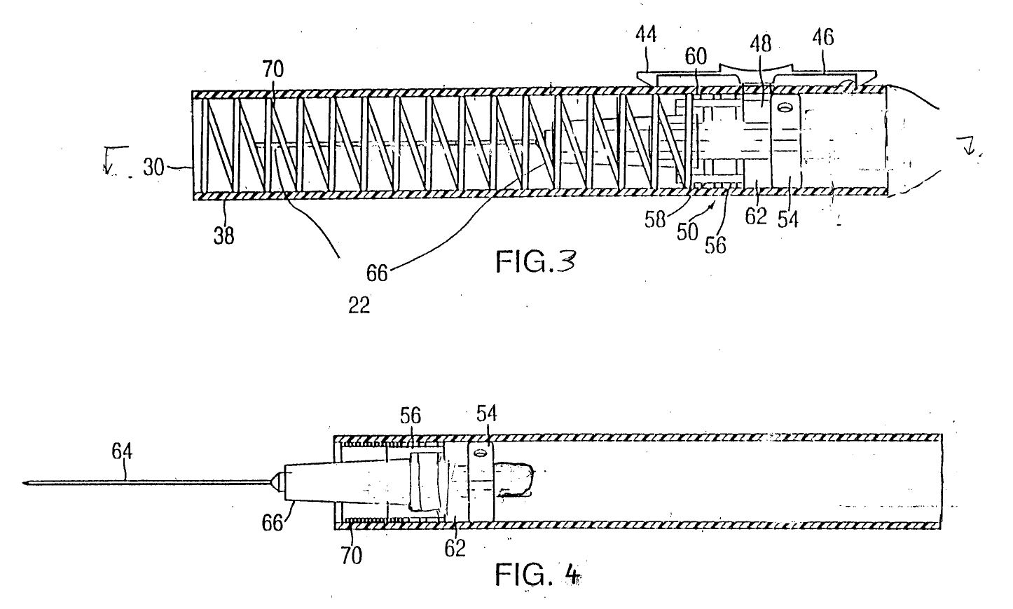

Referring now to FIGS. 3, 4 (in which the handle 34 and button 36 have been has been omitted for the sake of clarity) inside housing 24 there is a coupling 50. This coupling 50 has a circular disc 54 mounted coaxially on a shaft 52. Spaced axially away from disc 54 there is an adapter 56. The disc 54 and adapter 56 are separated by an annular space 62 having an axial length which is equal to, or slightly greater than the cross-sectional dimension of pin 60. The button 36 is positioned on the housing 24 so that its pin 60 extends into this space 62 as shown. Therefore the coupling 50 and the button 36 are effectively interlocked so that the coupling 50 can be moved in either direction along the length of the housing 24 and the button 36 will move with it (within the length of the slot 38) and vice versa.

Typically blade 22 is mounted on a base 66. The base 66 is secured to the adapter 56. The blade 22 may be permanently attached to the coupling 50. Alternatively, blade 22 could be removable as discussed in more detail below. For this latter configuration, the base 66 and the adapter could be shaped and constructed to allow the base to be removed from the adapted 50 at will, for example by using a bayonet-type or a Luer-lock type coupling.

Preferably, the end 30 is partially closed with a disc-shaped end wall 68. This end wall 68 has an opening (not shown) sufficiently large to allow the blade 22 and its base 66 to exit at least partially from housing 24, as seen in FIG. 4.

An optional compression spring 70 is provided between the end wall 68 and the coupling 50 in such a manner that normally the spring 70 pushes the coupling 50 and the blade 22 attached thereto away from wall 68. In other words, in this embodiment, the spring 70 urges the blade 22 toward its retracted position.

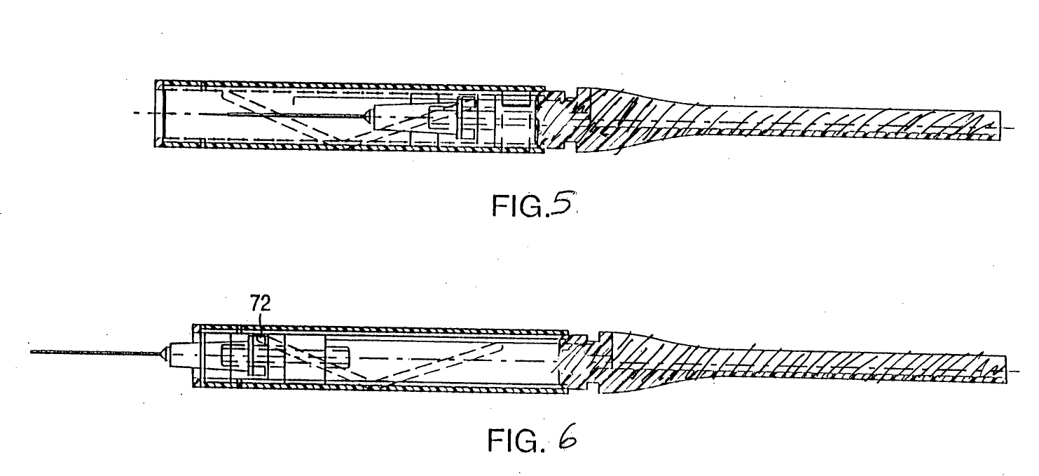

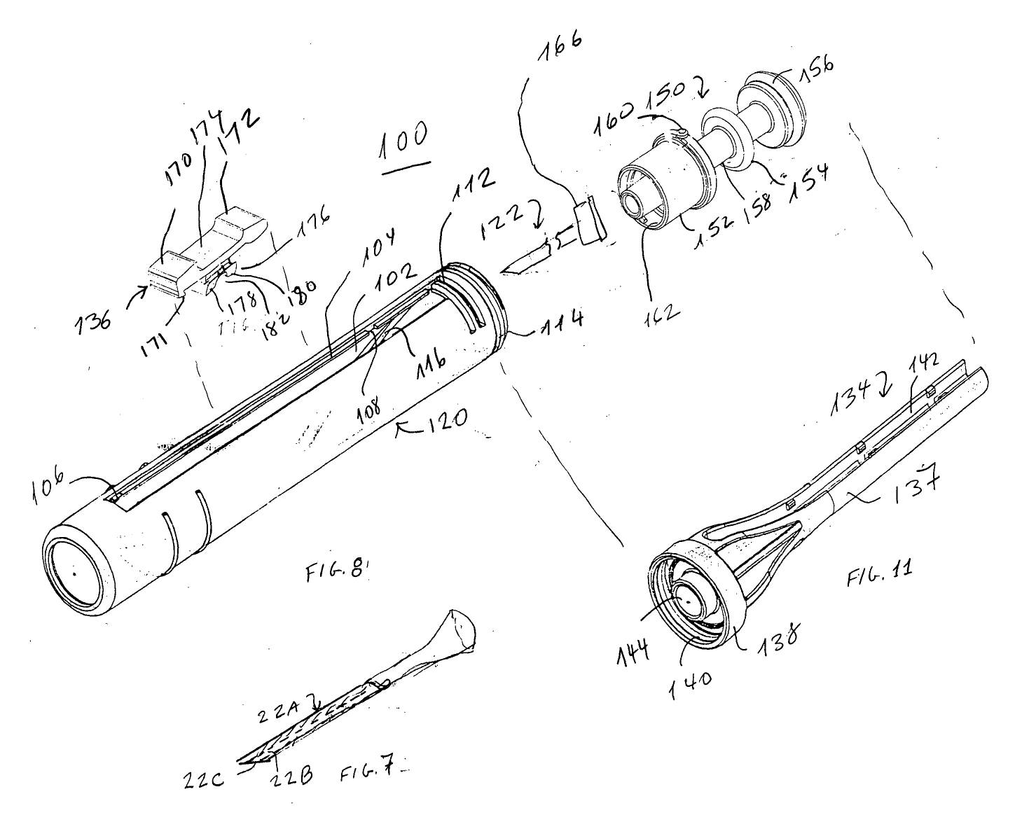

Yet in a further embodiment of the invention, shown in FIGS. 5 and 6 (in which the button 36 has been removed for the sake of clarity), the coupling 50 is provided with a boss 72 extending radially outwardly. In addition, the inner surface 26 of housing 24 is provided with a helical groove 74, with the boss 72 being seated with the groove 74 as shown. The coupling 50 and the blade 22 attached thereto are rotatable about the longitudinal axis of housing 24.

The operation of safety cutting tool 10 is now described in conjunction with the drawings. Initially, it is assumed that the tool 10 has a handle which has been provided with a spring 70 and groove 74. Prior to an injection the blade 22 is disposed in the handle 20 in its retracted position and is maintained in this configuration by the spring 70. In fact the tubing 18, handle 20 and blade 22 may come in a kit, several kits being made available for the operator, each blade having a different nominal size, including different length and/or width. Additionally, the kit may include other types of blades or cutting heads, such as a sleeve-type head formed with a cutting edge for biopsies. Particularly a cylindrical bladed cutting head can be affixed to the base so that tissue biopsies can be performed. One of these blades or cutting heads 22 is then selectively secured to the base 66, or directly to the coupling 50. Alternatively the scalpel 10 may be provided with a single blade 22 permanently mounted on base 66 or directly on coupling 50.

Preferably the handle 20 is sized and shaped so that it can held by the operator in the manner of a pencil with his forefinger resting on and pushing forward or backward on button 36. Initially, the blade 22 is latched in the retrieved position by the wind 46 engaging the rear end of the housing 24. Before it can be pushed forward, the blade must be unlatched or released. This can be accomplished by pushing down or radially inwardly on the button 36. This action causes the button to bend arms 44, 46 upward thereby releasing arm 46 from rear wall 32. The blade 22 is now free to move forward.

While the blade is translating, the blade 22 is rotating as it is reciprocated in and out of the housing 24. For this purpose, as the blade 22 is advancing with respect to the handle 20, the boss 72 rides in a caming action against the helical groove 74 and causes the blade to rotate. This combined advancement or translation of the blade and its simultaneous rotation is continued until the button 36 reaches the forward stop 40. At this point the forward wing 44 goes over the stop 40 and then forms an interference fit with it causing the button 36 and the blade 22 to lock in the extended position. This action is felt by the operator by a click and therefore the operator does not have to keep his eye on the handle or button 36. Since the blade 22 has now been locked in the extended position, the operator can release button 36 and start or continue with the use of the blade.

The blade can be used to immediately as it extends outwardly of the housing 24. Alternatively, the blade 22 can be used to cut as it is advanced axially forwardly as the button 36 reaches its forward or second position and is locked in place by the interference between the wing 44 and stop 40. When the button 36 is released from stop 40, the button 36, the coupler 50 and the blade 22 are forced to snap back into the housing 24 by the coil spring 70 until the button 36 reaches the rear wall 42. If a lock is provided at the rear wall, the button 36 locks onto the stop.

In an alternate embodiment the blade 22 only moves outwardly as the button 36, without any rotation. In order to stabilize the movement of the blade 22 and the coupling 50, one or more parallel grooves (not shown) may be provided along the inner surface 26. In order to avoid rotation, the button and the coupling 50 may be fixed or secured to each other. When the procedure with the blade 22 is finished, the operator can depress the button 36 again causing the wind 44 to disengage from stop 40. The spring 70 causes the blade 22 to rapidly be retrieved into the housing 24, again, without any rotation, until the button 36 reaches its initial position.

If the spring 70 is omitted, the blade 22 must be retrieved into the housing 24 manually using button 36.

The coupling 50 is which arranged and constructed to slidably engage the inner wall 28 of housing 24. Therefore, as the coupling 50 reciprocates within the housing in a smooth controlled movement without any radial jitter, thereby giving the operator a solid feel and ease of operation.

The scalpel 10 is shown in the previous Figures with a blade 22 that can be used to make incisions on tissues. Alternatively, the scalpel may be provided with a sleeve 22A as the cutting head and having an internal passageway 22B and a tip 22C which may be straight, or it may slanted as shown. In the sleeve 22A can be attached permanently and directly the coupling 50 or can be mounted on a base 66 just like blade 22. A scalpel equipped with a sleeve 22A can be used to perform biopsies. As the sleeve 22A is advanced and preferably rotated, it is automatically inserted into the tissues. The sleeve 22A is sharp around the tip 22C and cuts a cylindrical sample of the tissue that is lodged inside the passageway 22B. After the scalpel is removed, this sample is extracted.

Another alternate embodiment is shown in FIG. 8. In this Figure, a scalpel 100 is shown having a housing 120 with a button 136, a blade 122, a coupling 150 and a stem 134. An optional spring may be added in the housing to bias the blade 122 and coupling 150 as described above, however, it has been for the sake of clarity. These parts fit together in the same way as the parts of the scalpel 10 in FIGS. 1-6, however there are several structural differences between the two embodiments.

The housing is formed with a slot 102, having a pair of parallel ledges 104 (only one being visible in the Figure) running along the longitudinal edges thereof. At the forward end of the slot 102, the ledges 104 terminate short, so that the width of the slot at this location 106 is wider. Similarly, toward the rear end the slot a 102 there is provided another opening 108.

In addition, the housing is formed with several peripheral ribs 110 that form finger grips on the housing. Towards the rear of the housing 120, there are provided two ribs 112. The rear end of the housing 120 is formed with a helical thread 114. The housing 120 is formed with an inner helical groove 116.

The stem 134 has an elongated end 136 and a head 138 with an inner thread 140. The elongated end 136 is provided with a channel 142 that is in communication with a hole 144 in the head 138. The channel 142 and the hole 144 are not necessary for the present invention, as discussed below.

The coupler 150 has three axially spaced portions: a front portion 152, a center portion 154 and a rear portion 156. All three portions are disposed on a rod 158. Portion 154 is formed with a curved outer wall as shown. Portion 152 is formed on an outer wall with a boss 160 similar to boss 72 in FIG. 6. The portion 152 also has a cylindrical member 162 facing forward and defining a female section of a Luer lock. In the embodiment shown, the axle 158 is hollow, as described in more detail below.

Blade 122 includes a base 166 formed to define a male section of a Luer lock. In this manner, the blade 122 can be attached to the coupling 150 through the Luer lock formed by the base 166 and member 162.

The button 136 is formed with two winds 170, 172 separated by a flat portion 174. Wing 170 has at its forward end a downwardly extending tongue 171. Attached to the flat portion 174 is a downwardly extending tab 176 having two extensions 178 and 180. The two extensions 178, 180 define therebetween a semicircular opening 182.

The scalpel 170 is assembled by attaching the blade 122 on coupling 150, inserting the coupling into the housing from the rear and then attaching the stem 134 to the housing by threading the head 138 onto the threads 114. The button 136 is then mounted in the slot. The extensions 178, 180 are slightly longer than the width of the slot 102. Therefore, the button 136 is mounted by pushing it hard against the ledges 104 until the extensions snap through between the ledges. A portion of the extensions, as at 184, is narrower to accommodate the ledges 104. While the extensions 178, 180 are inserted into the slot 102, the coupling 150 must be positioned with its portion 154 disposed directly under the button 136 so that when the extensions 104 are pushed through, these extensions in effect trap the portion 154 with a radial section of portion 154 being disposed in between the extensions 178, 180. Once inserted, the button 136 is free to move longitudinally through the slot 102 in a smooth motion without substantial friction with the ledges 104. The button 136 drags the coupling 150 along, together with the blade 122.

As the button 136 approaches the rear of the housing, and the end of the slot 102, its wing 172 rides up and is forced radially away from the longitudinal axis of the housing, by the ribs 114. The winds 170, 172 and flat portion 174 are slightly flexible with respect to the tab 176, therefore, as the wing 172 rides on the ribs 112, it rotates slightly counter clockwise forcing the tongue 171 into opening 108, thereby locking the tab 176, and hence the coupling 150 and the blade 122 in place. In order to advance the scalpel, the user forces the button 120 forward, the tongue 171 to lift out of the opening 108. As the scalpel approaches the end of the housing, the tongue drops into the front opening 106 thereby locking the button to the forward position. At the same time, the blade 122 is carried forward until it extends outwardly of the housing to the extended position.

Thus, once assembled, the scalpel 100 operates the same way as the scalpel of FIGS. 1-6.

As mentioned above, commonly assigned U.S. Pat. No. 6,428,517, and incorporated herein by reference, discloses a safety syringe. More particularly the patent discloses a syringe having a handle identical to the housing shown in FIG. 8 with a needle coupled to the adapter 150 instead of a blade and a tubing passing through the channel 142 and hole 144 of stem 134 to provide a liquid to the needle through the adapter 150. Therefore an advantage of the embodiment of FIG. 8 is that the components shown in FIG. 8 can be used either with a cutting head (e.g., blade 122 or sleeve 22A), thereby forming the safety scalpel, or a needle and a tube (not shown in FIG. 8) thereby forming a syringe. Thus a manufacturer can make either syringes or scalpels almost the same components, thereby reducing its overall costs.

Preferably, once used, the whole safety scalpel is disposed. Since some health regulations require that disposable items with sharp tips or edges must be disposed separately. This can be easily accomplished by removing the adapter and attached cutting head from the housing (20,120) and then separating the cutting head from the adapter, for example by breaking the cutting head off.

In one embodiment of the invention, the safety scalpel is shipped without the button 36 being installed, and hence, the safety scalpel is effectively disabled because the cutting head is disposed inside the housing and cannot be advanced to extend outwardly of the housing. Just before use, the button 36, 136 is installed thereby rendering the safety scalpel useful.

After using the cutting head, the safety scalpel can be easily removed, and hence the safety scalpel can again be disabled.

For this purpose, the button 36 is constructed and arranged so that it is easily inserted into and/or removed or broken off from the housing 24. Normally, the button 36 is held in place by the tab 60 extending through the slot 38. The tab can be shaped and sized so that the button 36 can be lifted to pull the tab out of the slot 38. Alternatively, the button 36 can be made so it can easily sheared or broken off from the tab 60. Once the button 36 is separated from the housing 24, the connector 50 can no longer be moved through easily the housing.

Similarly the button 136 is formed so that its extensions 176 and 178 are readily insertible into the slot 102. After use, the button 136 can be pulled out and removed from the housing. Alternatively, the upper portion of the button 136, formed of the wings 170, 172 and flat portion 174 can be broken off and removed from the extensions 178, 180.

The rotation (if any) imparted to the cutting head (22, 22A, 122) is dictated by the pitch and length of the grooves 74 and 124 respectively. It has been found that preferably, the cutting head through its travel should rotate by about 20-270 degrees. More particularly, it has been found that the rotational movement is most effective when it covers about 180 degrees.

The subject invention provides the following advantages over the prior art:
A. Allows a round outer handle to be used that enables the operator to use a pen-like grasp that can be rolled between the thumb and fingers to enable easier to achieve curved incisions during movement of the blade.
B. Allows the cutting head to automatically retracted.
C. Allows the operator to advance the cutting head out of the housing sheath via manual operator control.
D. Allows the operator to use a pen-like grasp which has been shown to have superior tactile control.
E. Does not require extensive hand manipulation or the use of the second hand to retract the cutting head.
F. Allows for reuse of the cutting head on the same patient during a given procedure, but eliminates the risk associated with the re-use of a cutting head.
G. Allows a manufacturer to make several types of devices, such as a safety syringe, a blade or a narrow sleeve that can be used with the same housing, thereby reducing manufacturing costs, simplifying distribution and storage, etc.

This invention represents a major advancement in the safety and use of safety scalpels. It discloses a device that is easy and predictable in operation. The device minimizes or eliminates the risk potential of inadvertent blade sticks to health care workers.

In one aspect of the invention, a safety bladed device is presented that consists of a blade, a piston, a control tab, outer protective cylinder forming a housing. In one version a spring is used to bias the piston and provide automatic movement of the blade in relation to the outer cylinder. The control tab is a separate component enabling independent movement of the blade. The blade translates within the outer cylinder as the control tab maintains continuous and substantial control over the blade. In one embodiment, the control tab enables repeated protraction and retraction of the piston-blade assembly. In another embodiment, a permanent locking means is provided to lock the blade in place within the housing thereby disabling the device. In another embodiment, the blade simultaneously translates and rotates in relation to the outer cylinder while the control tab is translated in a linear fashion by an operator. In another version the piston assembly translates in a linear fashion while the control tab simultaneously translates in conjunction with the blade.

The control tab functions as an independent controller of the blade assembly in both versions. Locking of the blade within the housing is provided either by an interference fit between the control tab and the housing that prevents the movement of the blade, or by removing or separating the control tab from the housing. In both versions the piston-blade assembly remains encased in the outer cylinder.

While several embodiments have been described it should be understood that other modifications can be made to the invention without departing from its scope as defined in the appended claims.

1. A safety cutting tool scalpel comprising:
a tubular housing;
a coupling arranged for reciprocating movement within the housing between a first and a second position;
locking means for locking said coupling in at least of one of said positions; and
a cutting head attached to said coupling, said coupling cooperating with said housing to extend said cutting head outwardly of said housing in a cutting position when said coupling is in said first position and with said cutting head being disposed completely within said housing in a retracted position when said coupling is in said second position.

2. The tool of claim 1 wherein said housing is circular.

3. The tool of claim 1 wherein said locking means is adapted to lock said coupling with said cutting head being extended outwardly.

4. The tool of claim 1 further providing biasing means for biasing said cutting head toward said retracted position, whereby when said locking means is released, said cutting head is moved automatically to said retracted position.

5. The tool of claim 1 wherein said cutting head is permanently secured to said coupling.

6. The tool of claim 1 further comprising control means adapted for manually advancing said cutting head from said housing.

7. The tool of claim 6 wherein said control means is adapted to be selectively disabled thereby preventing said cutting head from being advanced.

8. A safety cutting tool comprising:
a cylindrical housing;
a cutting head disposed in said housing;
control means adapted to selectively move said cutting head between a retracted position in which said cutting head is completely disposed within said housing and an extended position in which said cutting head is operational to perform surgical procedures; and
biasing means for biasing said cutting head toward said retracted position.

9. The tool of claim 8 wherein said control means is selectively disabled with said cutting head in said retracted position to prevent said cutting head from being moved to said extended position.

10. The tool of claim 8 wherein said control means and said housing cooperate to selectively rotate said cutting head with respect to a longitudinal axis of said housing.

11. The tool of claim 10 wherein said housing and said control means cooperate to rotate and translate said cutting head along said longitudinal axis as said cutting head is moved between said retracted and extended positions.

12. The tool of claim 8 further comprising locking means for locking said cutting head in one of said positions.

13. The tool of claim 12 wherein said locking means is adapted to lock said cutting head in said extended position.

14. A tool cutting head comprising:
a cylindrical housing with an external wall, said external wall being provided with a longitudinal slot, said housing extending between a first and a second end walls, said first end wall being provided with an opening;
a coupling slidably disposed in said housing;
a cutting head connected to said coupling; and
a button extending through said slot and having a member engaging said coupling, said button cooperating with said coupling to selectively move said cutting head between a retracted position in which said cutting head is completely disposed in said housing and an extended position in which said cutting head extends outwardly through said opening, said cutting head being provided with locking means to selectively lock said cutting head in at least one of said positions.

15. The tool of claim 14 wherein said housing is provided with a helical groove and said coupling is provided with a boss, said helical groove and said boss cooperating to cause said cutting head to simultaneously translate and rotate said cutting head as said cutting head is moved between said positions.

16. The tool of claim 15 further comprising biasing means adapted to urge said cutting head toward said retracted position, whereby said cutting head automatically returns to said retracted position when released from said extended position.

17. The tool of claim 15 wherein at least a portion of said button is insertible and removable from said housing to thereby selectively enable and disable movement of said cutting head.

18. A safety scalpel comprising:
a cylindrical housing;
a cutting head reciprocating within said housing between a first position in which the cutting head is disposed within the housing and a second position in which at least a portion of the cutting head is disposed outside the housing; and
a control tab mounted on said housing and coupled to said cuffing head to reciprocate said cutting head between said positions.

19. The scalpel of claim 18 wherein said control tab is removable from said housing, whereby with said control tab removed, said cutting head is trapped within said housing.

20. The scalpel of claim 19 further comprising a biasing spring, said biasing spring urging said cutting head toward said first position.

21. The scalpel of claim 18 further comprising a first locking member for locking said cutting head in said first position.

22. The scalpel of claim 18 further comprising a second locking member for locking said cutting head in said second position.
